(19) United States
(12) Patent Application Publication
(10) Pub. No.: US 2025/0360247 A1
(43) Pub. Date: Nov. 27, 2025

(54) TREATED OSSEOUS PARTICULATE FLUFF COMPOSITION

(71) Applicant: Theodore Malinin, Key Biscayne, FL (US)

(72) Inventor: Theodore Malinin, Key Biscayne, FL (US)

(21) Appl. No.: 19/296,498

(22) Filed: Aug. 11, 2025

Related U.S. Application Data
(63) Continuation of application No. 18/971,812, filed on Dec. 6, 2024, now abandoned.
Application No. 18/971,812 is a continuation of application No. 16/922,422, filed on Jul. 7, 2020, now abandoned.

(51) Int. Cl. A61L 27/36; A61K 6/831

(52) U.S. Cl. CPC A61L 27/3608; A61L 27/3691; A61K 6/831; A61L 2430/02; A61L 2430/12; A61L 2430/38

(57) ABSTRACT

A method of making a bone graft composition is described wherein material including decalcified bone elongated particulate bone is dried in a constraining device or under pressure to produce a solid construct having dimensions corresponding to the constraining device or space to which the material occupied under pressure.

### CROSS-REFERENCE TO RELATED APPLICATIONS

The present application is a continuation of U.S. patent application Ser. No. 18/971,812, filed Dec. 6, 2024, which is a continuation of U.S. patent application Ser. No. 16/922,422, filed Jul. 7, 2020, the contents of both of which are hereby incorporated herein by reference in their entirety.

### TECHNOLOGY

The present disclosure is directed to bone compositions. More specifically, the present disclosure is directed to bone compositions comprising solid constructs formed from elongated particulate osseous tissue suitable for use in bone grafting procedures.

### BACKGROUND

Bone graft transplantation in dental applications includes ridge augmentations and obliteration of empty tooth sockets. If left untreated, the latter fills with clotted blood and granulation tissue and in a matter of months this material is replaced with new membranous bone. However, frequently the newly filled defect does not reach the previous, and adjacent ridge line. Infections and unforeseeable complications are common during this process due to generally contaminated mouth cavity.

Treatment options for such defects include packing the tooth defect with antiseptic or antibiotic impregnated material, synthetic or biologic materials including bone graft of particulate bone preparations, polymer based materials, block of bone, and plasma rich platelets. The most popular material is particulate bone allograft. However, these materials are difficult to retain. To alleviate this difficulty, particulate bone allografts are made into paste or past-like material. This necessitates mixing bone particles with substances such as glycerol, collagen, or other binding substances. These materials have disadvantages because matrices holding the particles are dense and make blood vessel ingrowth difficult to penetrate. Additionally, some of the substances such as glycerol have been found to produce undesirable reactions.

More recently demineralized bone particles have been used. These have an advantage of rapid release of bone stimulating factors. As with other particulate bone compositions, these particles are formed into putties, gels or pastes to improve handling characteristics of particulate bone but must be given time to heal before placement of tooth implants.

The present invention may be used to avoid or alleviate many of the above and other difficulties by providing a new construct based on the discovery of compact adherence of cortical bone fibers during freeze drying in a constraining container.

### SUMMARY

In one aspect, a compact biologically active bone graft material is provided that is configured to retain a tooth implant stem and produce rapid bone healing in other skeletal sites. Such bone graft material may be a bone graft composition constructed from one or more of allograft or xenograft bone. Such bone graft material may be a bone graft composition constructed from allograft bone, xenograft bone, or both. The bone graft composition may comprise a solid construct that has been dried under pressure.

In another aspect, a bone graft composition may be provided in a sterile package. The bone graft composition may comprise dried partially or wholly decalcified bone fluff particles, shards, or strands that have been freeze-dried or subjected to hypothermic dehydration in constraining containers or under pressure. In some embodiments, the bone graft composition is free or substantially free of bonding compounds. The bone graft composition may be self-sustaining. The bone graft composition may be structurally configured to retain metallic pins, screws or other configurations. The bone graft composition may have inductive capacity such that when implanted into human recipients new bone tissue is produced.

The bone graft composition may be made from partially or wholly decalcified cortical bone comprising bone particles that have been placed in water, saline or other aqueous solutions and then packed into a constraining device or placed under pressure between plates of various materials and then dried by freeze-drying or hypothermic dehydration to remove water content and bind the osseous material into a solid structure. In one example, the bone is placed into a freeze-drying chamber, under vacuum, to remove water and bind the osseous material into the solid structure. The bone particles may include elongated particulate bone such as bone shards, shavings, or fluff.

In another aspect, the present disclosure is directed to a method of preparing a bone graft composition comprising a solid construct. The method may include bone dried particles, under compression to yield a solid construct, wherein the bone graft composition comprises an allograft configured for human implantation. In another example, the bone graft may be a xenograft. In some examples, the bone graft composition includes one or more of autograft bone, allograft bone, or xenograft bone. The dried bone particles may be dried by hypothermic dehydration or freeze drying. The bone particles may include elongated particulate bone such as bone shards, shavings, or fluff.

The bone particles may be about 50% or less decalcified. In some embodiments, the bone graft composition comprises about 90 to about 100% decalcified bone compacted by a process of freeze-drying under self-induced normal or elevated pressure. In some embodiments, the bone graft materials utilized to construct the bone graft composition comprise 90 to 100% decalcified bone compacted by a process of freeze-drying or hypothermic dehydration under pressure.

### BRIEF DESCRIPTION OF DRAWINGS

The novel features of described embodiment are set forth with particularity in the appended claims. The described embodiment may be best understood by reference to the following description taken in conjunction with accompanying drawings in which:

FIG. 1 is a method of making a bone graft composition according to various embodiments described herein;

FIG. 2 is a photograph of cortical bone fluff or shards before decalcification according to various embodiments described herein;

FIG. 3 is a photograph of decalcified cortical bone fluff particulates freeze-dried under compression to yield solid construct bone graft compositions of various shapes and dimensions according to various embodiments described herein;

FIG. 4 is a photograph of decalcified cortical bone particles comprising bone particles freeze-dried under compression to yield a solid construct bone graft composition in the form of a sheet according to various embodiments described herein; and

FIG. 5 is a photograph under magnification of the bone graft composition of FIG. 4 showing the decalcified bone particles bound together by the process of freeze-drying under compression according to various embodiments described herein.

### DESCRIPTION

Bone regeneration occurs if native bone regeneration mechanisms are provided with space into which to grow. Thus, an implanted matrix or scaffold into which bone may grow has been used to promote bone regeneration. Regeneration includes absorption or replacement of the implant by the newly regenerated bone. Bone grafts may be used to replace, augment, or repair bone and other structural features in animals including humans. Traditionally, bone grafts are implanted during a bone grafting procedure in which the bone graft is placed adjacent to or into native bone.

Osteoinduction is the process that induces osteogenesis or new bone formation by osteoblasts and may include signaling or induction by growth factors. Osteoinduction includes native cell recruitment and stimulation of osteoblastic cell differentiation cascades. Osteoconduction generally describes a surface phenomenon in which bone growth occurs on a surface. For example, bone grafts preferably comprise implant materials having biocompatible characteristics suitable for osteoconduction. Stable anchorage of bone grafts may be referred to as osseointegration and may include direct contact between bone and the implant graft or metal implant such as a screw.

According to various embodiments, the bone graft materials, methods, and constructs are configured for implantation into an animal. When implanted, the implanted bone graft of the present invention is configured to provide superior osteoconduction when compared to osteoconduction contributions of conventional implants or bone grafts. For example, the implant may be configured to guide reparative growth of natural bone in a manner such that bone regeneration results in quicker healing or earlier onset of denser bone regions when compared to conventional implants or bone grafts.

Techniques for tissue preparation vary significantly. Among the ways to obtain and prepare bone graft materials is demineralization of structural bone. However, several deficiencies of current bone grafts, such as bone allografts, remain unaddressed. These deficiencies include porosity attributed to the intertrabecular space constituting a major portion of the graft, lack of uniformity, lack of osteoconduction, and lack of mechanical support. Various embodiments of the present disclosure are specifically configured to obviate these deficiencies to improve implantation and grafting outcomes. For example, bone graft compositions described herein may be compacted to provide solid bone graft constructs. The construct as described herein may be configured to accommodate and retain a metal screw which has been inserted without damage to the construct.

In various embodiments, the bone graft is or comprises a treated bone composition of osseous tissue particulates made from decalcified cortical bone obtained from a mammal.

The osseous tissue may be harvested from long, flat and/or other bones of a mammal, which may include multiple mammals. Exemplary mammals include but are not limited to a primate, a bovine, a porcine, or an equine or other mammalian animal. In one embodiment, the mammal is a human.

The osseous tissue may be harvested postmortem and then dehydrated. The osseous tissue may be harvested from shafts of long bone or cortical tables of flat bones of a mammal. When dehydration is complete, the material is said to be dried. Drying may include any suitable drying method, such as freeze-drying.

The cortex of the dried, harvested osseous tissue may be particulated to generate bone particles of suitable dimensions. In one example, bone particles may be generated from the cortex of dried bone using a bone shard collector, plane, drill or any such similar device. The bone particles may comprise elongated bone particulates such as shards, strands, bone fluff, and/or shavings. The bone particles may include branched and/or fragmented strands and/or fluff, for example. The bone particles preferably have micron scale dimensions, but may include length dimensions greater than micron scale.

The osseous tissue may be decalcified. Decalcification may include partially decalcified or wholly decalcified bone. Typically, decalcification will follow particulation of the osseous tissue to generate bone particles. However, in some embodiments, decalcification may occur prior to particulation. If material is prepared by other than aseptic methods, bone particles (e.g., shards, fluff, fibers or shavings) can be immersed into a sterilizing solution or be sterilized by other methods such as exposure to ethylene oxide gas, irradiation or other methods. The sterilizing solution may include Dakin solution or other solution which deactivates prions.

The decalcified bone particles may be subjected to a rehydration process. Rehydration may comprise contacting the bone particles with an aqueous solution. For example, the bone particles may be immersed in water or another aqueous solution.

The rehydrated bone particles may then be packed in a constraining device of desired dimensions and subjected to hypothermic dehydration or freeze-drying.

Upon completion of the drying process, the now condensed bone composition retains a desired shape and/or dimensions of the constraining device in which it was constrained during drying. Beneficially, no bonding material is needed as drying the rehydrated decalcified elongated bone particles under compression results in a binding of the bone particles to form a solid construct. In some embodiments the bone composition comprises 90% to 100% decalcified bone.

The bone graft composition may be utilized as an allograft or xenograft, as the case may be, in bone grafting procedures. While such a situation would likely be unique, in some embodiments, the bone graft composition may include autograft bone. Thus, the bone graft composition may comprise a bone construct preparation made of bone graft materials consisting of or comprising decalcified bone particles. The decalcified bone particles may be partially or wholly decalcified. The bone particles may be or comprise elongated particulated bone comprising shards or fluff freeze-dried under compression. The bone graft composition may consist of or comprise a particulate bone graft preparation made of decalcified bone particles held together by their ability to form solid construct due to the ability of the bone particles to bind together when freeze-dried in a constrained environment with self-induced compression. In some embodiments, the bone particles may be or comprise elongated particulated bone comprising shards or fluff dried by hypothermic dehydration under compression. The bone graft composition may comprise a particulate bone graft preparation made of bone graft materials consisting of or comprising decalcified elongated bone particulates, such as shards or fluff, held together by their ability to form solid construct due to the ability of the bone particles to bind together when dried by hypothermic dehydration or freeze-drying in a constrained environment with self-induced compression. In various embodiments, the bone graft composition may comprise an allograft bone preparation specific to mandibular or maxillary defect and retention of dental implants.

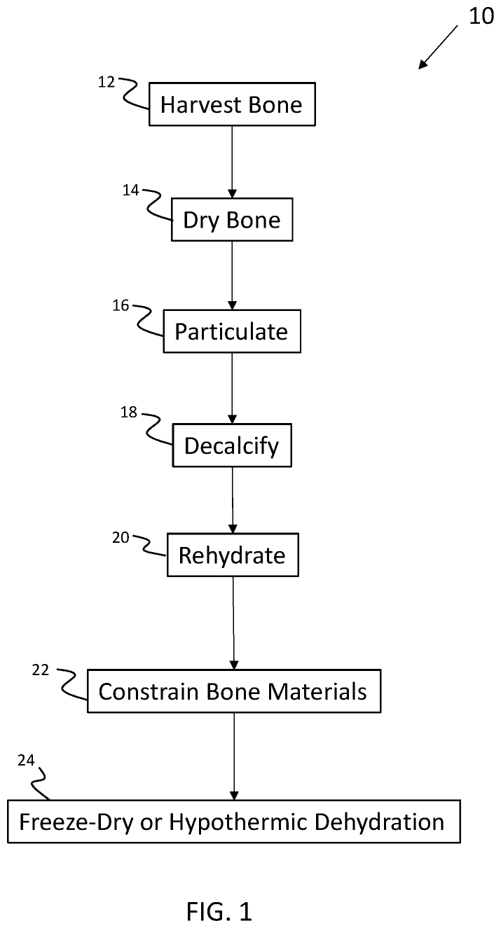

FIG. 1 depicts a method 10 of making a bone graft composition according to various embodiments.

In various embodiments, the method 10 of making the bone composition comprises harvesting osseous tissue 12. The osseous tissue may be harvested from shafts of long bone or cortical tables of flat bones of a mammal. The osseous tissue may typically be harvested postmortem or from surgically removed bone specimens. In some embodiments, the mammal is one of a primate, an equine, a bovine, a porcine or other mammalian animal. In one example, the primate is a human. In one embodiment, the osseous tissue is harvested from a human postmortem, wherein the bone is excised aseptically without muscle tendon or fascial attachments. Thus, in one example, only periosteum is left on the excised bone.

The osseous tissue may be dried 14 prior to decalcification 18 and/or particulation 16 to remove water content. Drying may be by any known bone drying method. For example, the osseous tissue may be dried 14 by freeze-drying, hypothermic dehydration, without freezing, or by chemical dehydration. Drying will typically reduce water content to less than about 10% or preferably about 6% or less residual moisture.

The method may also include particulating 16 the osseous tissue to generate bone particles. The bone particles may include elongates bone particulate, which may also include or be referred to as bone strands, shards, shavings, fragments, or bone fluff. In some embodiments, the bone particles may comprise truncated bone particulates in addition to or instead of elongated bone particulates. The bone particles may be branched and/or fragmented.

Bone particles having a largest dimension smaller than about 50 microns do not adhere tightly to each other. Round microparticulate bone also does not adhere tightly. Likewise, larger pieces of bone do not form a compact mass to produce the desired bone composition. In various embodiments, the bone graft composition comprises less than about 20%, less than about 15%, less than about 10%, less than about 5%, less than about 2.5%, less than about 1%, less than about 0.5%, or is devoid of bone particles having a particle size less than about 50 microns, round microparticulate, or large pieces of bone.

Accordingly, particulation 16 may include generating bone particles. Bone particles may be or include elongated particulate bone. Elongated particulate bone may have height and width dimensions between about 50 microns and about 1 mm or an average particle diameter, excluding length dimension, greater than about 50 microns. For example, elongated particulate bone may have a particle size between about 100 microns to about 500 microns, about 100 microns and about 750 microns, 100 microns and about 1 mm, about 300 microns and about 700 microns, or about 500 microns to about 1 mm. The elongated particulate bone may have length dimensions greater than about 1 mm longer. In one example, the elongated particulate bone utilized for the bone graft composition preferably has length dimensions between about 1 mm and about 7 mm. However, longer length dimensions may be used such as between about 7 mm and about 20 mm, or greater. In one embodiment, the elongated particulate bone or elongated particulate bone portion of the bone particles of the bone graft composition has an average length dimension greater than about 1 mm, about 2 mm, about 3 mm, about 4 mm, about 5 mm, about 6 mm, about 7 mm, about 8 mm, about 9 mm, about 10 mm, about 15 mm, or about 20 mm. In one embodiment, the bone graft composition includes an elongated particulate bone portion having an average length between about 2 mm and about 5 mm and average height and width dimensions greater than about 50 microns and less than about 1 mm. For example, the bone graft composition may include elongated particulate bone having micron scale height and width dimensions and length dimensions of between about 1 mm and about 7 mm or greater. The elongated particulate bone preferably has length dimensions greater than height and width dimensions. Elongated particulate bone may be fragmented and/or branched. The bone shards and/or fluff may be branched, fragmented, or both. For example, the bone graft composition may include elongated particulate bone wherein between about 10% and 100%, about 20% and about 40%, about 40% and about 60%, about 60% and about 80%, about 80% and about 100%, about 20% and about 80%, about 40% and about 80%, or greater than about 50% by weight are branched, fragmented, or both.

In some embodiments, at least about 70%, about 75%, about 80%, about 90%, about 95%, about 98%, about 99%, or about 100% of bone by weight in the bone graft composition is provided by elongated particulate bone. In embodiments wherein the bone graft composition includes additional bone particles in addition to elongated particulate bone the remaining bone particles in the bone graft composition may comprise bone particles having dimensions outside the dimensions described herein with respect to the elongated particulate bone.

Elongated particulate bone may include shards. Shards may include bone fragments having an average particle diameter between about 500 micron and about 1 mm, such as between about 600 microns and about 800 microns. Bone shards may have an average particle length greater than about 1 mm, such as between about 1 mm and about 10 mm, about 1 mm and about 7 mm, or about 2 mm and about 5 mm. In some embodiments, greater than about 80%, greater than about 85%, greater than about 90%, greater than about 95%, greater than about 98%, or about 100% of bone shards in the bone graft composition have an average particle diameter between about 500 micron and about 1 mm, such as between about 600 microns and about 800 microns and an average particle length greater than about 1 mm, such as between about 1 mm and about 10 mm, about 1 mm and about 7 mm, or about 2 mm and about 5 mm. Bone shards may be branched or fragmented.

Elongated particulate bone may include bone fluff. Bone fluff may include strands of bone having an average particle diameter between about 50 microns and about 500 microns, such as greater than about 100 microns or between about 100 microns and about 500 microns. Bone fluff may comprise micronized strands of bone having an average particle length greater than about 1 mm, such as between about 1 mm and about 10 mm, about 1 mm and about 7 mm, or about 2 mm and about 5 mm. In some embodiments, greater than about 80%, greater than about 85%, greater than about 90%, greater than about 95%, greater than about 98%, or about 100% of bone fluff in the bone graft composition has an average particle diameter between about 50 microns and about 500 mm, such as between about 100 microns and about 500 microns, and an average particle length greater than about 1 mm, such as between about 1 mm and about 10 mm, about 1 mm and about 7 mm, or about 2 mm and about 5 mm. Bone fluff may be branched or fragmented.

The bone shards and/or fluff may be branched, fragmented, or both. For example, elongated particulate bone may comprise shards and/or fluff wherein between about 10% and 100%, about 20% and about 40%, about 40% and about 60%, about 60% and about 80%, about 80% and about 100%, about 20% and about 80%, about 40% and about 80%, or greater than about 50% by weight are branched, fragmented, or both.

In various embodiments, the bone graft composition comprises elongated particulate bone or an elongated particulate bone portion comprising between about 0% and about 100% bone shards. In these or other embodiments, the bone graft composition comprises elongated particulate bone or an elongated particulate bone portion comprising between about 0% and about 100% bone shards. For example, the bone graft composition may comprise elongated particulate bone or an elongated particulate bone portion at various ratios of bone shards to bone fluff by weight, such as about 1 to about 9, about 2 to about 8, about 3 to about 7, about 4 to about 6, about 5 to about 5, about 6 to about 4, about 7 to about 3, about 8 to about 1, or about 9 to about 1.

In an example, bone particles have a particle size greater than 50 microns and the strands, shards or fragments have the length in the range of 1 to 7 mm. In a further example, the bone particles have a particle size between about 100 microns and about 500 microns and the length of branched strands or fragments is between about 2 mm and about 5 mm. In one embodiment, the osseous tissue may be cut with a bone shard collector to form dried, e.g., freeze-dried, bone strands or fluff which may be branched or fragmented.

In some embodiments, the bone particles may include truncated particulate bone having micron scale height and width dimensions. For example truncated particulate bone may include bone particles having height and width dimensions or average particle diameters as described above and elsewhere herein with respect elongated particulate bone and length dimensions less than about 1 mm. For example, truncated bone particles may have a particle size between about 100 microns to about 500 microns. The truncated particulate bone preferably has length dimensions greater than height and width dimensions. Truncated particulate bone may be fragmented and/or branched.

The bone graft composition may include bone particles comprising elongated particulate bone and/or truncated particulate bones, as described herein. For example, elongated particulate bone may comprise between 0% and 100% of the bone particles by weight. In these or other examples, truncated particulate bone may comprise between 0% and 100% of the bone particles by weight. In some embodiments, at least about 70%, about 75%, about 80%, about 90%, about 95%, about 98%, about 99%, or about 100% of bone by weight in the bone graft composition is provided by elongated particulate bone and truncated particulate bone. For example, the bone graft composition may include bone particles wherein elongated particulate bone and/or truncated particulate bone provide at least about 75%, about 80%, about 85%, about 90%, about 95%, about 98%, about 99%, or about 100% of bone in the bone graft composition by weight. In one example, the elongated particulate bone has an average length dimension greater than 1 mm or between about 1 mm and about 7 mm and the truncated particulate bones have a particle size between about 50 microns and about 1 mm or between about 100 microns and about 500 microns. In embodiments wherein the bone graft composition includes additional bone particles in addition to elongated particulate bone and/or truncated particulate bones the remaining bone particles in the bone graft composition may comprise bone particles having dimensions outside the dimensions described herein with respect to the truncated particulate bones and elongated particulate bone.

In various embodiments, the bone graft composition comprises an elongated particulate bone portion and/or a truncated particulate bone portion. In one example, the elongated particulate bone portion provides between about 0% and about 100% of the bone of the bone graft by weight. In one example, the truncated particulate bone portion provides between about 0% and about 100% of the bone of the bone graft by weight. In one example, the bone graft composition comprises bone particles wherein the weight ratio of truncated particulate bones to elongated particulate bone is about 1 to about 9, about 2 to about 8, about 3 to about 7, about 4 to about 6, about 5 to about 5, about 6 to about 4, about 7 to about 3, about 8 to about 1, or about 9 to about 1.

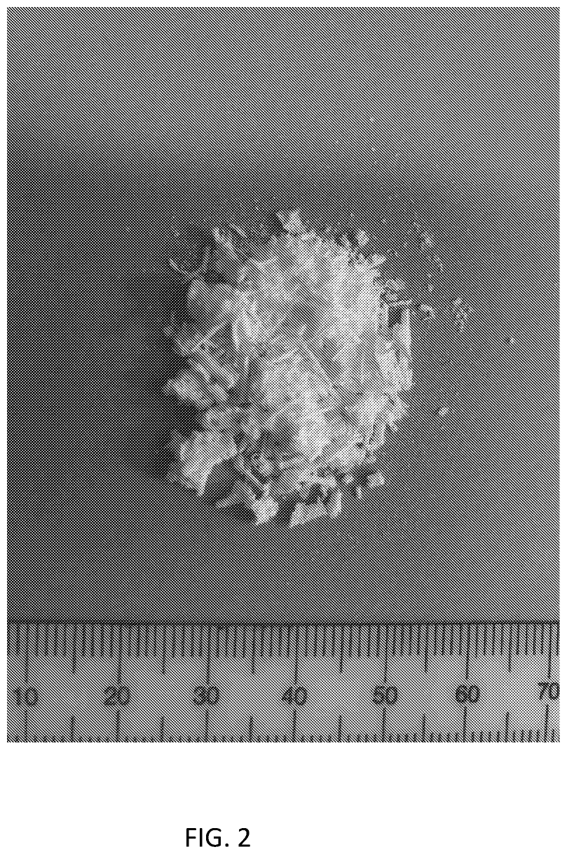

FIG. 2 illustrates an example of particulated cortical bone prior to decalcification where the bone particles comprise fluff and shards.

The dried bone particles may be stored prior to decalcification. Storage may be at room temperature.

Other currently less preferred methodologies may also be employed. For example, in some embodiments, harvested osseous tissue may be cut into pieces larger than micron scale prior to drying 14 and/or decalcification 18 and thereafter particulated 16. For example, the osseous tissue may be cut into pieces from dried, e.g., freeze-dried, osseous tissue prior to being exposed to a decalcifying solution during decalcification 18. The pieces may be further particulated 16 to generate bone particles after drying 14, decalcification 18, and/or rehydration 20 to generate bone particles. In one embodiment, the osseous tissue is particulated after decalcification 18 or rehydration 20.

The dried osseous tissue may be decalcified 18 with a suitable decalcifying solution. Decalcification 18 may refer to wholly or partially decalcified bone. The osseous tissue may comprise dried bone particles generated during particulation 16. Decalcification 18 may include contacting the osseous tissue with a decalcifying solution to achieve partial decalcification or surface decalcification of the bone.

The decalcification solution may include an acid treatment or other decalcification agent. For example, a suitable decalcification solution may include hydrochloric acid, citric acid, ethylenediaminetetraacetic acid (EDTA), nitric acid, trichloroacetic acid, formic acid, Plank-Rychlo solution, Morse's solution, or a combination thereof. In one embodiment, a chelating decalcifying solution is used comprising EDTA with 0.07% to 10% glycerol or ethylenediaminetetraacetic acid (EDTA)/tris(hydroxymethyl)aminomethane(Tris)-hydrochloride. In various embodiments, the decalcifying solution comprises at least one of IN HCl; citric acid, 5% to 20% w/v; 0.24M disodium or tetrasodium salts of EDTA in a balanced salt solution, saline, or water neutralized to a pH around 6.8 to 7.2 with NaOH; and a mixture of 5M EDTA and 5M citric acid. The solution may be agitated to increase contact or coverage. Agitation may also encourage movement of the solution and reduce localized stagnation, bubbles, or variations.

Decalcification times may vary with different decalcifying solutions. In one example, decalcification 18 may be performed in about 10% to 20% of bone particle to HCl by weight at ambient temperature for a time period not exceeding about 15 minutes. However, other combinations of ratios of particles to solution, acid or other decalcifying agent concentrations, and temperatures may be used to achieve the desired extent of decalcification.

Decalcification 18 of the osseous tissue may be performed to reduce calcium content by 50% or greater. For example, decalcification may be performed to reduce calcium content greater than about 55%, greater than about 60%, greater than about 65%, greater than about 70%, greater than about 75%, greater than about 80%, or greater than about 85% of the pre-decalcified calcium content. In one embodiment, following decalcification 18, the osseous tissue is 90% to about 100% decalcified. Remaining percent of residual calcium may be determined by standard methods such as inductively coupled plasma emission spectroscopy.

The method may also include rehydrating 20 the decalcified osseous tissue. Rehydration 20 may include contacting, e.g., submerging, all or a portion of the decalcified bone with an aqueous solution. Non-limiting examples of suitable rehydration solutions include water, saline, and other aqueous solutions.

Following rehydration 20, the rehydrated decalcified osseous tissue may be constrained 22 in a constraining device and dried 24. As described above, the rehydrated decalcified osseous tissue may comprise bone particles particulated prior to or following rehydration. The bone particles may be reconfigured via the combination of constraining 22, e.g., compression or under pressure, and drying 24, e.g., freeze-drying or hypothermic dehydration, to form bone graft compositions comprising solid constructs of various sizes and dimensions. For example, rehydrated bone particles may be packed into a constraining device and subjected to drying. The constraining device may include constrained dimensions with respect to the rehydrated bone particles. In one embodiment, the constraining device may apply external pressure to or compress the rehydrated bone particles. While constrained, the bone particles may be dried to bind the osseous material into a solid structure.

A constraining device may comprise a vessel or other structure having constrained dimensions. Compression may be performed manually when the constraining device is packed. In some embodiments, the constraining device may include a mechanism by which pressure or compression may be applied to the bone materials. A portion of the water content may be expressed from the bone particles by squeezing the preparation. During the drying process, additional internal or self-induced compression is produced by the material itself as it is being dried. Self-induced compression may include compression in the constraining device produced by the material being dried. The compression may result in tight association between bone particles to form a bone graft composition comprising a solid construct.

The constrained bone particles may be dried 24 to generate a solid construct. Drying 24 may include hypothermic dehydration, without freezing, freeze-drying, or by chemical dehydration. Hypothermic dehydration, for example, may employ a technique comprising drying the bone particles in a vacuum at temperatures from about 1 to about 20 degrees centigrade. Freeze-drying may employ a technique comprising drying the bone particles in a vacuum at temperatures below 1 degree centigrade. The bone particles and constraining device may be placed in a drying chamber. In one embodiment, the drying chamber is configured for freeze-drying wherein the bone particles are subjected to low temperature and vacuum pressure to remove water and bind the osseous material into a solid structure. In another embodiment, the drying chamber is configured for hypothermic dehydration wherein the bone particles are subjected to low temperatures above 0 degrees centigrade and vacuum pressure to remove water and bind the osseous material into a solid structure.

Drying is preferably to between about 6% to about 10% of residual moisture. Drying to greater than about 10% residual moisture may result in weaker association between bone particles and a less solid bone composition, while further drying to less than about 6% residual moisture may result in alteration of biomechanical properties of the bone material.

It will be appreciated that the method may include additional or fewer steps. For example, the method may include subjecting rehydrated compressed decalcified bone particles to freeze-drying or hypothermic dehydration to form a solid construct.

The bone particles that are dried while constrained are preferably micron scale particles as described above. The desired particle size may be generated during particulation to achieve micron scale dimensions. In some embodiments, bone particles may include strands or fluff having microscale dimension length dimensions as described above approximating about 1 mm to about 7 mm. In some examples, strands or fluff may be fragmented or branched.

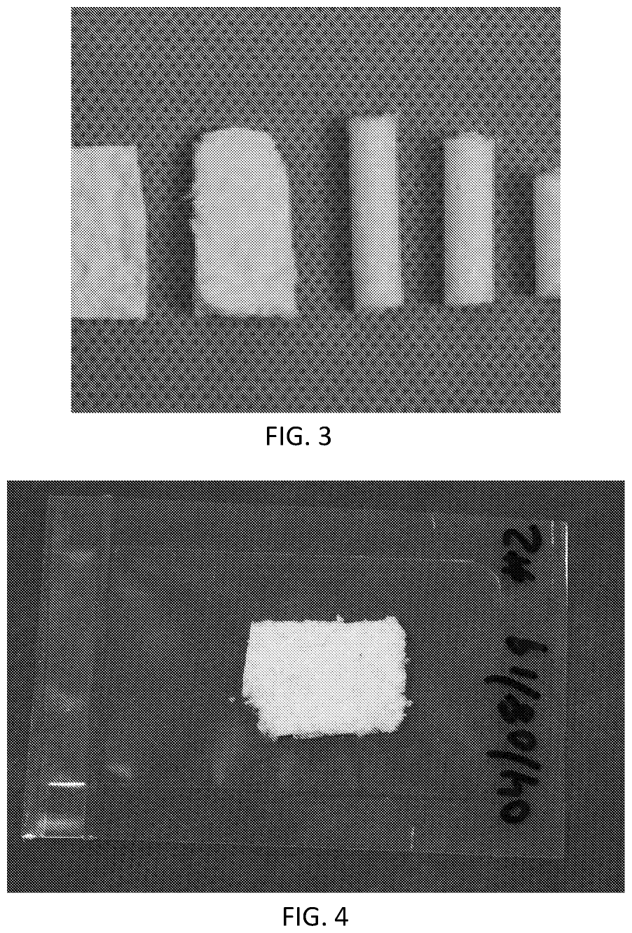

Constraining devices may include or be configured to include dimensions within which the bone composition may be constrained to form solid constructs of matching dimension. For example, constraining devices and corresponding constructs formed therein may include any desired size and shape such as cuboidal, cylindrical, spherical, oblong, arcuate, or other regular or irregular geometric shape. In various embodiments, the constraining device includes a round and rectangular plastic tube (e.g., polypropylene, teflon, nylon, tygon, butyrate, tritan, etc.) having perforated walls to allow water vapor to escape. Embodiments may include constraining devices configured to form dimensions matched with a desired dimension corresponding to that of a grafting site of the graft recipient. FIG. 3 exemplifies various bone compositions comprising decalcified bone fluff freeze-dried under compression to yield flat sheet 20, rectangular composition 22, and tabular compositions 24, 26, 28.

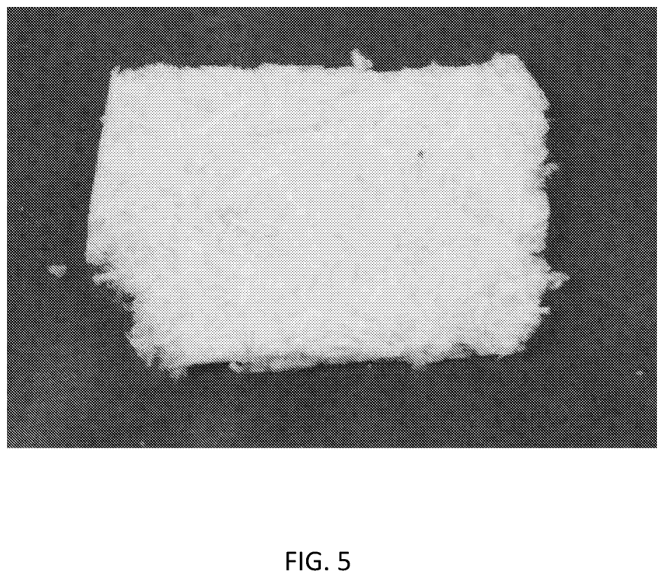

The constraining device may include a volume or container into which the bone material may be packed such that the particles are compressed together for drying. The constraining device may comprise a mold into which the bone materials are placed and compressed together for drying. In one embodiment, the constraining device comprises two opposed surfaces that compress the bone particles during drying to form a bone graft composition comprising a bone sheet. For example, the bone particles may be compressed between plates of various materials and then placed into a freeze-drying chamber, under vacuum to remove water and bind the osseous material into a solid construct. FIG. 4 exemplifies a bone composition 30 comprising partially decalcified bone particles bond together to form a sheet. The bone particles comprise bone shards that were dried in a constraining device comprising two perforated cellophane sheets. Freeze-drying was accomplished while the bone shards were placed between the perforated cellophane sheets and weight was applied to the same. FIG. 5 is a photograph of the bone composition 30 shown in FIG. 4 under magnification.

Drying while constrained or under pressure as described herein produces a bone graft composition comprising a solid construct that retains structure without requiring bonding materials.

In various embodiments, the bone graft construct comprises about 80% to about 100% decalcified bone by weight, including residual water weight, such between about 90% and about 100%, as greater than about 91%, greater than about 92%, greater than about 93%, greater than about 94%, greater than about 95%, greater than about 96%, greater than about 97%, greater than about 98%, or greater than about 99% decalcified bone. In an above or a further embodiment, the bone graft composition may comprise between about 90% to about 100% decalcified bone particles by weight. Thus, in at least one embodiment, the bone graft composition consists of decalcified bone dried under pressure.

In various embodiments, the bone particles subjected to constrained drying, include between about 50% and about 100%, such as between about 60% and about 100%, about 70% and about 100%, about 80% and about 100%, about 90% and about 100%, or between about 85% and about 100% bone particles having height and width dimensions or average diameter greater than about 50 microns and length dimensions of about 1 mm or greater.

The bone graft composition may include additional materials within the construct. In one example, the bone composition comprises only decalcified bone particles and residual moisture. In some embodiments, the bone graft composition may also include added supplemental or fortifying components osteogenic bone factors, antibiotics, or other therapeutic agents.

Current bone constructs for implantation require bonding materials such as glycerol, collagen, fibrin, hydroxyapatite, calcium sulfate, polyether ketone (PEEK), and osteocell cellular bone material. The methodology described herein for making a bone graft composition allows formulation of a solid construct without addition of bonders thus making bonders unnecessary. In some embodiments, the bone graft composition may be bonder free or substantially bonder free. In an example, the bone graft composition comprised only decalcified bone particles, residual moisture, and non-binder materials. In another example, the bone graft composition comprises less than 7%, less than 5%, less than 2%, less than 1%, less than 0.5%, less than 0.1%, less than 0.01%, or less than 0.001% bonding material by weight.

In one embodiment, the bone graft composition comprises bone particles within a container. The bone particles may be dried and partially or wholly decalcified as described herein. The bone particles within the container may be packaged sterile. The bone particles may be dry. The bone particles may be freeze-dried or dried by hypothermic dehydration.

In one embodiment, the bone graft composition comprises a sterile package of dried partially or wholly decalcified bone fluff particles, shards, or strands freeze-dried in constraining containers or under pressure. The bone construct may be free or substantially free of bonding compounds. The bone construct may be self-sustaining. The bone construct may have structural integrity to retain metallic pins, screws or other configurations. The bone construct may also have inductive capacity, when implanted into human recipients, to produce new bone tissue.

In one some embodiments, bone graft materials comprising particulated bone may be provided in a dried and partially or wholly decalcified state wherein a user may add aqueous solution to rehydrate the bone particles followed by drying of the hydrated bone particles while constrained. In one example, the bone graft materials may be provided in a container having desired dimensions for shaping of the bone construct or may be transferred to another container having desired dimensions of the bone construct for drying in the compressed or shape conformed condition.

The bone graft construct may be utilized in various bone grafting procedures. For example, mandibular and maxillary bone loss is common, as are problems with dentition, and frequently result in tooth loss or necessitate tooth extraction. Some embodiments of the bone graft composition may be particularly configured for use in connection with dental procedures. That is, the herein described bone graft compositions may be configured for use as restorative dental materials and in restorative dental procedures. Bone grafts compositions may be used in a surgical treatment connection with other implant materials, such as a dental implant, tooth implant, or prosthetic. For example, some such embodiments are configured for use in connection with dental procedures comprising dental or tooth implantation, restoration of lost bone, e.g., ridge augmentation, as well as obliteration of tooth sockets following tooth extraction. In some instances, benefits of extraction site grafting according to the present embodiments may include preservation of bone contour for dental implants, denture stability, soft tissue aesthetics, and maintaining periodontal status of adjacent teeth. For example, grafting into extraction sites may be used to restore gum or jaw following tooth extraction or loss due to congenital defects, periodontal disease, or infections or trauma, for example, or may reduce negative sequela to permit dental implants to be placed in a position that is ideal for function and aesthetics. A graft ridge may also be employed to improve denture stabilization, support, and retention.

In various embodiments, the present embodiments include methods, systems, and apparatuses for preparation, manufacture, and supply of bone graft and bone graft material. For example, in one embodiment, a system and method of preparing a bone composition comprises preparing a bone allograft specific for a particular tooth in the maxilla and the mandible. In some embodiments, for example, an empty tooth socket may be obliterated with a rapidly healing implant material, which may be or include the bone graft composition to provide, retain, or at least partially support a stem of a tooth implant. In one embodiment, the bone graft composition may be further configured to secure itself by osteointegration and may include biological or synthetic materials such as a gel or metal, e.g., a polymer, hydrogel, or biologically compatible metal. In one embodiment, the bone graft composition comprises a solid construct with one or more surface features, ridges, or perforations. The bone graft composition may be formed or shaped to a customized dimension using manual, mechanical, or electronic instrumentation based on three-dimensional modeling of an implant site.

In one embodiment, a method of using the bone graft composition comprises implanting the bone construct into a human recipient where the bone graft composition initiates response by forming new bone. For example, the bone graft composition may initiate a cellular response of host cells that includes reprogramming of host cells. Host cells may infiltrate the construct and therein spread throughout to form infiltrating host tissue. For example, new host cells may grow between bone particles of the bone graft composition. The infiltrating cells may exhibit and express osseous markers with a capacity to initiate differentiation into osseous lineages. In one example, the infiltrating host cells associated with the implanted bone graft composition express the marker Sox 9 for osteogenic differentiation. Biological activity may be promoted by partially decalcified cortical bone particles of the bone graft composition when implanted due to release of BMP at accelerated rates compared to other commonly implanted bone constructs. In one embodiment, the bone graft composition includes a body comprising surface dimensions or features, such as grooves, threads, perforations, or ridges, defined on a surface thereof.

Among the suitable applications for certain bone compositions described herein are mandibular and maxillary ridge augmentations and rapid obliteration of tooth socket defects, e.g., in the maxilla or mandible. The bone grafts of present invention can be also used in skeletal grafting such as spinal fusions, obliteration of bone defects, fracture healing and other applications

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or reorganizing any of the disclosed steps, components, elements, features, aspects, characteristics, limitations, and the like, of the various non-limiting and non-exhaustive embodiments described in this specification.

Various elements described herein have been described as alternatives or alternative combinations, e.g., in a lists of selectable actives, ingredients, or compositions. It is to be appreciated that embodiments may include one, more, or all of any such elements. Thus, this description includes embodiments of all such elements independently and embodiments including such elements in all combinations.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” refers to “x”, “y”, or both “x” and “y”, whereas “either x or y” refers to exclusivity.

Any numerical range recited herein includes all values and ranges from the lower value to the upper value. For example, if a concentration range is stated as 1% to 50% or between 1% and 50%, it is intended that values such as 2% to 40%, 10% to 30%, 1% to 3%, or 2%, 25%, 39% and the like, are expressly enumerated in this specification. These are only examples of what is specifically intended, and all possible combinations of numerical values and ranges between and including the lowest value and the highest value enumerated are to be considered to be expressly stated in this application. Numbers modified by the term “approximately” or “about” are intended to include+/−10% of the number modified.

What is claimed is:

1. A method of making a bone graft composition, the method comprising:
generating a self-sustaining solid construct by drying a mixture of bone particles in water, saline, or other aqueous solution while in a constraining device or under pressure, the solid construct substantially free of bonding materials and having structural integrity to retain metallic pins and screws,
wherein the bone particles comprise at least partially decalcified cortical elongate bone shards and/or fluff having a length between about 1 mm and about 10 mm and a diameter between about 50 microns and about 500 microns,
wherein the solid construct includes at least 70% by weight of the at least partially decalcified cortical elongate bone shards and/or fluff,
wherein the at least partially decalcified cortical elongate bone shards or fluff were cut from dehydrated bone tissue comprising shafts of long bone or cortical tables of flat bones before being at least partially decalcified in a decalcifying solution, and wherein the bone particles including the at least partially decalcified elongate bone shards and/or fluff were hydrated in the water, saline, or other aqueous solution prior to the drying in the constraining device or under pressure.

2. The method of claim 1, wherein the bone tissue was dehydrated using freeze-drying, hypothermic dehydration, without freezing, or chemical dehydration.

3. The method of claim 1, wherein the bone tissue was harvested from shafts of long bone or cortical tables of flat bones of a mammal.

4. The method of claim 3, wherein the bone tissue was harvested postmortem or from surgically removed bone specimens.

5. The method of claim 4, wherein the mammal is one of a primate, an equine, a bovine, or a porcine.

6. The method of claim 5, wherein the primate is a human.

7. The method of claim 6, wherein the human postmortem bone was excised aseptically without muscle tendon or fascial attachments.

8. The method of claim 1, wherein the bone particles were dried in the constraining device.

9. The method of claim 8, wherein the dehydrated cortical bone tissue was cut with a bone shard collector, plane, drill, lathe or similar device to form at least the at least partially decalcified cortical elongate bone shards and/or fluff of the bone particles.

10. The method of claim 9, wherein the at least partially decalcified cortical elongate bone shards and/or fluff are branched and fragmented.

11. The method of claim 1, wherein the solid construct is free of bonding material.

12. The method of claim 1, wherein the bone particles are at least 50% decalcified.

13. The method of claim 1, wherein drying the mixture of hydrated bone particles comprises freeze-drying.

14. The method of claim 1, wherein drying the mixture of hydrated bone particles comprises drying by hypothermic dehydration.

15. The method of claim 1, wherein drying the mixture of hydrated bone particles comprises drying to between 10% and 6% residual moisture by weight.

16. The method of claim 15, wherein the bone tissue was dehydrated to less than 10% residual moisture by weight.

17. The method of claim 1, wherein the bone particles further comprise partially and/or wholly decalcified rehydrated bone particles comprising micronized particles processed to form a cellular bone matrix.

18. The method of claim 1, wherein the bone particles further comprise truncated bone particles having an average particle size greater than 50 microns and an average length dimension less than about 1 mm.

19. The method of claim 1, wherein the solid construct includes at least 90% by weight of the at least partially decalcified cortical elongate bone shards and/or fluff.

20. The method of claim 1, wherein the bone particles were dried under pressure.
